(19) United States
(12) Patent Application Publication
(10) Pub. No.: US 2005/0090725 A1
(43) Pub. Date: Apr. 28, 2005

(54) Disposable couplings for biometric instruments

(76) Inventors: Joseph Page, La Jolla, CA (US); James Plante, Del Mar, CA (US)

Correspondence Address: Joseph Page, PO Box 757, La Jolla, CA, 92038, US

(21) Appl. No.: 10/695,358

(22) Filed: Oct. 28, 2003

(51) Int. Cl. A61B 5/00

(52) U.S. Cl. 600/344

(57) ABSTRACT

Photoacoustic measurement system are configured with a special view towards efficient coupling of optical and acoustic energy between respective transducers and a tissue test site. In particular, a disposable substrate provides support for advanced optical paths including, for example, windows, lenses, and index matching gels or fluids. In addition, substrates may also accommodate arrays of coupling sites corresponding to a plurality of acoustic detectors spatially separated. These substrates may additionally include means to affix and secure the device to a measurement head having optoelectronic and electromechanical transducers therein. Further, these substrates include mechanisms which help to affix the substrates to test sites in stabile and secure fashion.

### BACKGROUND OF THE INVENTIONS

1. Field

The following invention disclosure is generally concerned with biometric measurement systems employing a photoacoustic effect and specifically concerned with improved coupling between instruments and human tissue.

2. Prior Art

Photoacoustic spectroscopy is a mature body of science. When applied to living beings, or in vivo photoacoustic spectroscopy, the technique involves coupling various transducers to live tissues. In these specialized systems, optical sources and acoustic detectors are placed in communication with various parts of the anatomy. This gives rise to a great plurality of coupling techniques each having associated benefits and faults. The following examples illustrate some important techniques used to join human tissues with electronic measurement systems.

A first example of special coupling between a spectroscopic system and living human tissue relates to a principle call Attenuated Total Reflection, so called ‘ATR’. In U.S. Pat. No. 6,430,424 by Berman et al, light is made to repeatedly interact with skin tissue on a finger which is placed in intimate contact with a specialized waveguide or beam guide. In this way, the active ‘cross section’ is increased from more typical single pass techniques.

Inventor Geva presents a special acoustic cell of spherical section in U.S. Pat. No. 6,466,806 dated Oct. 15, 2002. This cell is distinctive because it includes an optical port (lens) in which light may be introduced into the cell; and further, the cell is optically transparent whereby light may propagate to pass through the acoustic cell. Tuned acoustic cells promote an effect sometimes call ‘resonant photoacoustic spectroscopy ’.

Some human organs require still further special coupling consideration. For example, one might directly probe the eyeball. Oraevsky et al teach approaches in U.S. Pat. No. 6,405,069, entitled: “Time-Resolved Optoacoustic Method and System for Noninvasive Monitoring of Glucose”. In these systems, a laser and a pressure transducer are put into contact with the eye via the sclera. An interface at the sclera is formed when a solid element is placed in contact therewith. Some users find such application a bit uncomfortable.

A mineral oil immersion system is taught in U.S. Pat. No. 6,567,688 which is an electromagnetically induced thermoacoustic tomography system based upon microwaves.

Professor Lilienfeld-Toal presents an excellent system for photoacoustic spectroscopy involving mid-IR wavelengths in U.S. Pat. No. 6,484,044 dated Nov. 19, 2002. A bulk material is placed in direct contact with the skin. Acoustic waves propagating in the tissue pass therefrom and into the bulk material where they are collected. An acoustic transducer is attached to the bulk material whereby it picks up acoustic energy. The bulk material further provides that optical energy be transmitted therethrough and into the test tissue.

Andrew C. Tam presents photoacoustic techniques in his Rev. Mod. Phys., paper Vol. 58, No. 2 Apr. 1986 entitled “Applications of Photoacoustic Sensing Techniques”. In this teaching, a coupling gas permits propagation of both optical excitation energy and acoustic return signals.

While systems and inventions of the art are designed to achieve particular goals and objectives, some of those being no less than remarkable, these inventions have limitations which prevent their use in new ways now possible. Inventions of the art are not used and cannot be used to realize the advantages and objectives of these inventions taught herefollowing.

### SUMMARY OF THE INVENTIONS

Comes now, James Plante and Joseph Page with inventions of disposable couplings for biometric instruments which may be worn on the body. It is a primary function of these systems to provide secure and efficient couplings which may be comfortable when worn. These may include the special case where the instrument is to be worn for extended periods of time.

Photoacoustic measurement systems typically require a test site be energized with optical (photonic) energy and require sensing of returned mechanical (acoustic) energy. To effect such, electronic transducers including lasers and microphones must be interfaced with tissue. As made clear from the prior art, the physical interface may be embodied in many forms. In inventions taught herein, wearable systems which couple to a users' skin surface are of immediate concern. In particular, these systems include at least one laser and at least one microphone which are put into close proximity with a test subject's skin surface via coupling means.

Lasers and microphones may be included as parts of a system measurement head. Since a microphone which might include a relatively smooth surface is to be in communication with skin which is sometimes irregular and rough, a joint formed by the microphone and skin might not be conducive to efficient transmission of mechanical energy therebetween. In some cases, a medium such as a thick gel with appropriate density might be used to fill pores and spaces natural to skin surface and promote better transmission of acoustic energy into a microphone transducer. Carefully prepared gel materials facilitate transmission of energy to and from a tissue being tested.

Similarly, laser light tends to scatter from the irregular surface of the skin and is not well coupled to tissue lying just below the skin surface. Special geometries which may include special optical lenses, including possibly an immersion lens, may be used to encourage better transmission of laser light into tissue test sites.

Finally, where a measurement head is to be placed into contact with a test site, it is preferable that motion between the test site and detector head is minimized. An affixing means such as adhesive or mechanical grit helps to anchor a test head to a skin surface in a spatial coupling.

Laser and microphone devices tend to be expensive but are long-lasting and remain functional over a long lifetime. As such, they are preferably permanently affixed and mounted within an appropriate housing herein referred to as a ‘measurement head’. Conversely, materials such as gels, fluids, and lenses are sensitive to exposure and may have short lifetimes as a result of becoming dirty and otherwise contaminated. It is therefore desirable to separate these into cooperating subsystems whereby one is made disposable but renewable, and the other is permanent. Together the subsystems cooperate to form an improved comprehensive device with greater coupling efficiency and accordingly better system signal to noise ratio and ultimately higher performance.

A disposable element is prepared with optical coupling means, acoustic coupling means, and mechanical coupling means. The disposable element may be detached from a test head and replaced with a fresh and renewed disposable element from time-to-time as necessary. In this way, the lasers and microphones of a wearable in vivo photoacoustic effect measurement system are made efficiently coupled to tissue operable by users with special facility and equipment.

Objectives of these Inventions

It is a primary object of these inventions to provide couplings between advanced biometric measurement systems and living human tissue.

It is an object of these inventions to provide systems which pass optical and acoustic energy between tissue and electronic transducers.

It is a further object to provide clean, sanitary and disposable systems for use in biomedical measurement.

A better understanding can be had with reference to detailed description of preferred embodiments and with reference to appended drawings. Embodiments presented include particular ways to realize these inventions and are not inclusive of all ways possible. Therefore, there may exist embodiments that do not deviate from the spirit and scope of this disclosure as set forth by the claims, but do not appear here as specific examples. It will be appreciated that a great plurality of alternative versions are possible.

### BRIEF DESCRIPTION OF THE DRAWING FIGURES

These and other features, aspects, and advantages of the present invention will become better understood with regard to the following description, appended claims and drawings where:

FIG. 1 is a perspective exploded view drawing of an article of these inventions in relation to its environment of use;

FIG. 2 illustrates a first side of these articles and its relationship with a detector or measurement head;

FIG. 3 is a perspective drawing showing another side of a disposable coupling element and its relationship with a test site on human tissue;

FIG. 4 is a cross sectional block diagram showing most important elements of a disposable coupling element;

FIG. 5 is a block diagram provided with principles of optical coupling in isolation for more clarity;

FIG. 6 shows acoustic coupling isolated in cross section;

FIG. 7 illustrates one example of mechanical coupling with a detector head and mechanical coupling with a tissue test site;

FIG. 8 is the sum of the three previous views together;

FIG. 9 is a cross section diagram of alternative forms of these inventions;

FIGS. 10 and 11 similarly illustrate alternative versions;

FIG. 12 is a perspective drawings of yet another version;

FIG. 13 is a cross sectional drawing of the same version of that in FIG. 12; and

FIG. 14 is a cross sectional view of a similar version.

Glossary of Special Terms

Throughout this disclosure, reference is made to some terms which may or may not be exactly defined in popular dictionaries as they are defined here. To provide a more precise disclosure, the following terms are presented with a view to clarity so that the true breadth and scope may be more readily appreciated. Although every attempt is made to be precise and thorough, it is a necessary condition that not all meanings associated with each term can be completely set forth. Accordingly, each term is intended to also include its common meaning which may be derived from general usage within the pertinent arts or by dictionary meaning. Where the presented definition is in conflict with a dictionary or arts definition, one must use the context of use and liberal discretion to arrive at an intended meaning. One will be well advised to error on the side of attaching broader meanings to terms used in order to fully appreciate the depth of the teaching and to understand all the intended variations.

Disposable—is meant to mean inexpensive items with relatively short lifetime suitable for frequent replacement in a system having more durable elements.

Photoacoustic Spectroscopy—is a measurement field whereby a target is illuminated with an optical signal to produce a thermal or mechanical stress wave. Analysis of the acoustic return signal yields information about the target site.

Optical source—an optical source is an optoelectronic device suitable for generating optical energy in response to electrical stimulation. For purposes of these inventions, this includes diode emitters and lasers. In particular, optical sources used herein include semiconductor lasers where an ‘optical source’ may be comprised of several or one laser.

Measurement head—a measurement head refers to an arrangement of transducers and accompanying support electronics which is integrated together in a compact package suitable for mobility and convenient application to test sites of interest.

Detectors—include acoustic or audio detectors sometimes and commonly known as microphones. A detector may include an array of individual elements spatially distributed and arranged in cooperation with each other.

Acoustic Conduit—an acoustic conduit is any path which conducts propagation of mechanical energy via pressure waves. In some cases, it is a preferential path whereby energy flows efficiently in the conduit but less efficiently in surrounding space.

“Coupling Means”

For purposes of these inventions, ‘coupling means’ is anything arranged to place a first member in better relation to a second member. This is especially the case when energy is passed from a first member to a second member; but not necessarily the case. Although in many instances, a coupling means increases the efficiency in which energy may be transferred, sometimes a coupling means may be arranged to maintain a spatial relationship between two members.

A ‘coupling means’ therefore can be said to promote a specific function. Many alternate forms of coupling means may be used to accomplish an identical task. A particular coupling means employed in one version may be chosen for a particular application at hand. However, the essence of these inventions is not changed by the particular choice. Therefore various versions should not be limited to one particular type of coupling means because others which achieve identical or equivalent function may be equally valuable. The limitation defined by ‘coupling means’ is met when the coupling function is effected. Therefore, by use of the term ‘coupling means’ it is meant that any conceivable means for suitable coupling as described is anticipated. Experts will recognize that there are many thousands of possible ways of bring about a coupling means and it will not serve a further understanding of these inventions to attempt to exhaustively catalogue them here. The reader will appreciate that the broadest possible definition of ‘coupling means’ is intended here.

Some examples of ‘coupling means’ include the following:

Optical Coupling Means . . .

An ‘optical coupling means’ couples optical energy from an optical source to a target. An optical coupling means may include common optical elements such as windows, lenses, thin film anti-reflection coatings, et cetera, and further may include free space optical paths, index matching fluids, as well as optical paths of complex composition such as volumes of human flesh.

Acoustic Coupling Means . . .

‘Acoustic coupling means’ couples acoustic energy from a test site to a detector, and sometimes in the opposite direction. An acoustic coupling means may include elements such as acoustic resonance cells, high density materials having a low acoustic impedance, index matching gels, among others.

Spatial Coupling Means . . .

‘Spatial coupling means’ are configured and arranged to hold a first element in positional or spatial relation with a second element.

These terms which are functional in nature may be used throughout this disclosure including the claims. One should remain aware that any particular means which may be later provided as an example is not meant to limit the ‘means plus function’ to that example but rather the example is provided to further illustrate certain preferred possibilities. Thus the ‘means for’ or ‘means plus function’ should not be limited to any particular structure which may be called out but rather to any matter of causing the function described to be effected. The reader will recognize it is the function to be carried out which is the essence of these inventions and many alternative means for causing the function to occur may exist without detracting from any combination or combinations taught as part of these inventions.

### PREFERRED EMBODIMENTS OF THESE INVENTIONS

In accordance with each of preferred embodiments of these inventions, there is provided disposable coupling articles for use in conjunction with photoacoustic measurement systems. It will be appreciated that each of these embodiments described include an apparatus and that the apparatus of one preferred embodiment may be different than the apparatus of another embodiment.

These inventions are primarily defined as disposable couplings in photoacoustic biometric measurement systems, the devices being formed of a thin substrate element having an optical coupling to efficiently pass optical energy from an optical source into human flesh and an acoustic coupling to efficiently pass acoustic energy to/from an acoustic transducer into human flesh.

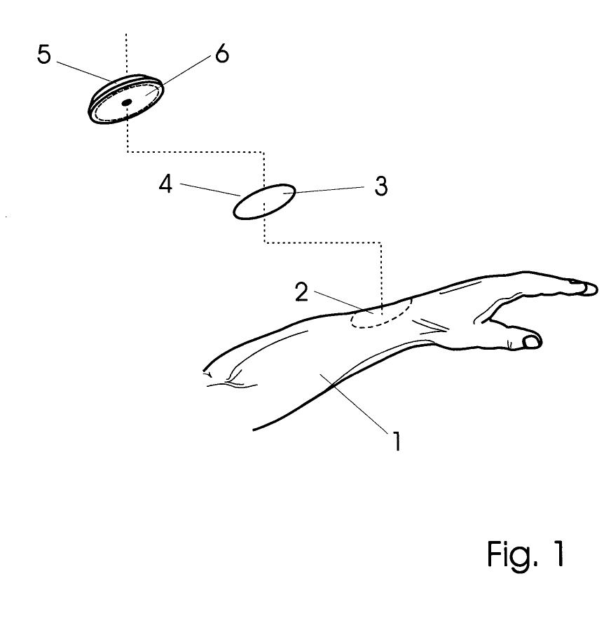

With reference to drawing FIG. 1, one can more fully appreciate the environment and use of preferred versions. A human subject 1 to be tested includes tissue at a test site 2 at the top side of the wrist where the skin is moderately thick and access to blood in the tissue is available. A thin wafer or coupling substrate in the form of a disk shaped element includes a bottom side 3 and a top side 4 as well as a circular periphery. The bottom side is configured and arranged to directly engage the skin tissue surface at the test site, while the top side is configured and arranged to cooperate and interface with a measurement head 5 at its bottom side 6. Accordingly, the substrate forms an interface between the detector and the tissue. When the measurement head is brought into contact with the disposable coupling and further into contact with the test site, the system readily permits the transfer of optical energy from the measurement head through the coupling and into the tissue; further, the system readily permits the transfer of acoustic energy in the tissue to pass through the coupling and into the measurement head. The coupling is arranged to promote this energy transfer. In addition, the coupling may also include a function to maintain the position of the measurement head with respect an arbitrary spot on the tissue, i.e. a stable and unchanging test site.

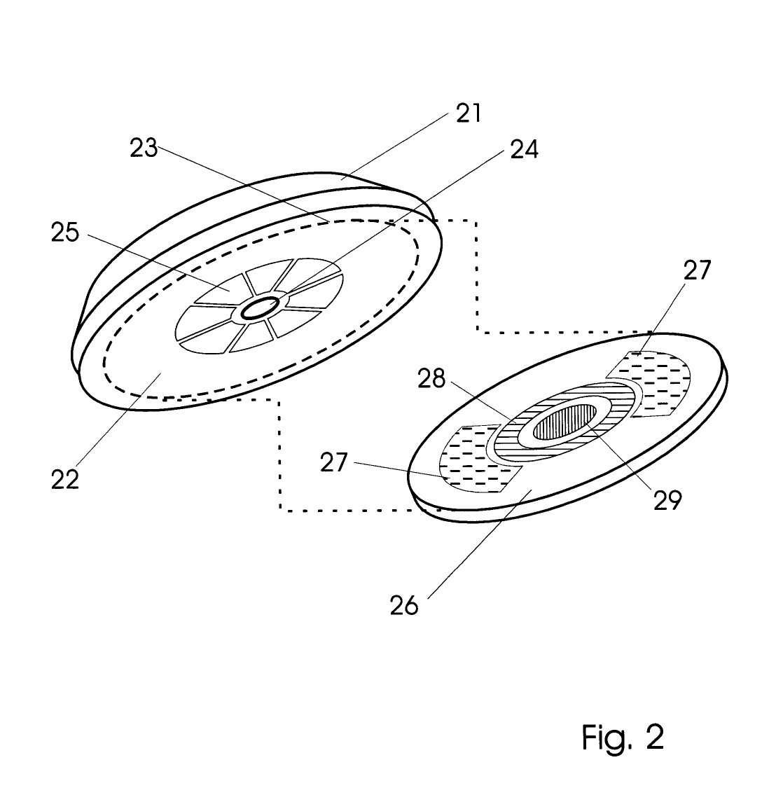

More complete detail is shown in the perspective drawing FIG. 2 where a measurement head 21 and one surface, the top surface, of a disk shaped coupling element are presented in relation to each other. The underside 22 of the detector head may include mechanical means of indexing and receiving cooperative parts, for example a disposable coupling, therein. For example a surface relief ridge 23 may be provided such that a similarly arranged disk shaped element may be fit therein a cavity formed by the bottom side surface and the ridge. Further, a detector head may expose special transducers such as at least one laser optical source, via port/aperture 24 is shown in the diagram, and at least one microphone, an array 25 of piezoelectric acoustic detectors is shown in the diagram. This particular array is shown as an eight element detector array symmetric about an axis in the shape of an annulus. Other array shapes are possible and fully anticipated, but convenience suggests the concentric symmetry shown for clarity. Supporting electronics such as energy supply and driving electronics may be found in the measurement head but away from the bottom side surface and those are not illustrated in these diagrams. A coupling element 26 of inexpensive and disposable material may support a various regions thereon each having a different coupling function. For example, a first region 27 is a pair of identical areas which might be simply used to adhere the top of the disk to the measurement head at its bottom surface via common adhesive agents having removable properties. Coupling disks can be prepared where an appropriate adhesive is applied in the regions designated for such. When pushed to the measurement head, the adhesive causes the coupling disk to remain attached and affixed and more importantly aligned with the measurement head. In this way, the other regions of the coupling disk maintain a specified positional relationship with the measurement head. Region 28 might include a special gel material applied to the top surface of the coupling disk. That gel may be suitable for transmitting acoustic energy to and from materials in which it might contact. The gel can be applied to the disk in a thin film and it can be thick and sufficiently viscous whereby it tends to remain in its predetermined location on the disk and tends not to migrate about the surface thereof. In some preferred versions, an annular shaped region of acoustic coupling gel substantially matches the shape of an array of acoustic detectors 25. When the coupling disk is pushed into contact with the measurement head, the acoustic transmitting gel comes into contact with the microphones of the array. Further, the adhesive holds the coupling disk to the measurement head and thereby the shaped coupling gel to the array. Region 29 forms an optical window operable for permitting efficient transmission of light beams therethrough. Light beams generated in the measurement head may exit therefrom at port/aperture 24. These beams being in particular alignment with coupling disk region 29, an optical window, are permitted to pass orthogonally through the coupling disk and exit the other side. Certain versions of coupling disks may include anti-reflection coatings, lenses, index matching fluids, among others, in the region indicated as 29.

A coupling disk element may be formed of hard plastics for example. Molding processes lend extraordinary latitude with respect to manufacturing and design advantage. Further, plastics are inexpensive and highly workable and compatible with many chemical and biological materials. Thus, coupling disks of these inventions are generally made from plastic or plastic-like materials. While the coupling element's shape is not essential to functionality, and it is appreciated that a rectangular shape could similarly be configured with these three coupling functions, preferred versions will take a similar shape as a measurement head and these might preferably be round in cross section; i.e. a coupling ‘disk’.

As described, coupling disks may include thereon materials such as adhesives, gels and fluids. As such, they may be sensitive to exposure and contamination. Manufactured coupling disks may therefore be package in individually wrapped packets to protect the devices prior to use. In proper use, coupling disks may be removed from their packages and pushed to a measurement head before being applied to a test site on a tissue surface.

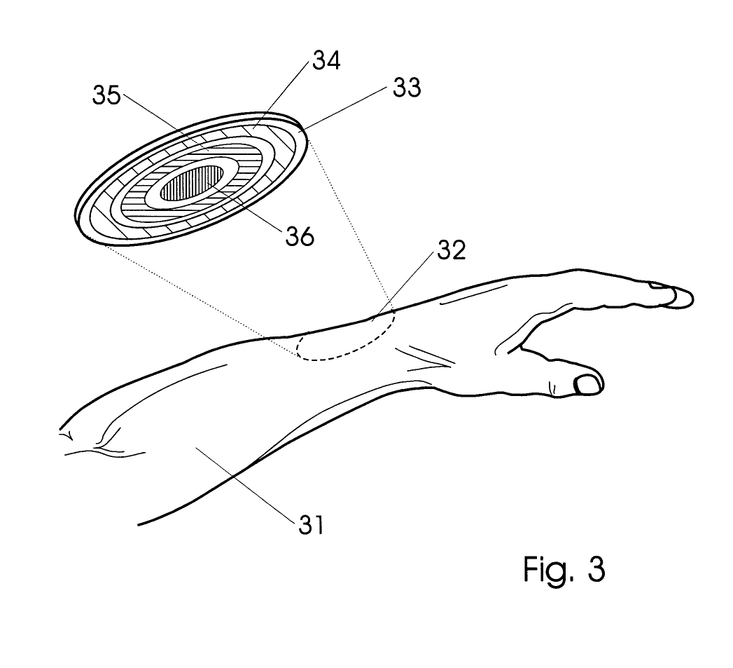

FIG. 3 illustrates another side of the disposable coupling element, a side which couples to a tissue test site. Living human 31, having test site 32 at the surface of skin tissue may be joined by one surface of a coupling element 33. The bottom surface of the coupling disk may include three specially prepared regions, each to effect particular functionality. In this example, each region concentric with the other having circular symmetry and the two outer regions form annuli. An outer region 34 may be arranged to engage and adhere to the skin surface. Certain adhesives are appropriate for use on the human skin and may effectively hold the coupling disk steady and fast to the skin surface. When the device is to be removed, the adhesive yields to peeling pressure without excessive pain as a result of pulling the skin. An alternative to adhesive include merely a mechanical gripping agent such as micro teeth form in the plastic of the disk surface. The region chosen for the spatial coupling means is selected to be at the periphery of the device to gain leverage for the task at hand. A distributed bond between the coupling element and the skin is most effective for promoting stability and a reliable bond or grip.

Another region 35 of the coupling element lies inside the first on a smaller annulus. This region may be arranged to cooperate with the one identically shaped on the opposite side of the coupling element. Both regions are arranged to include dense fluid or gel medium which promotes propagation of acoustic waves. The gel makes good and certain acoustic contact with the skin surface which tends to be porous. It is not necessary that the gel used for acoustic energy transfer on the top of the coupling element be the same as that on the bottom. The gel on the top provides acoustic coupling between two objects with smooth surfaces, i.e. the disk and the microphone array. However, the gel used for acoustic coupling on the bottom surface must provide acoustic coupling with the rough and porous surface of the skin. As such, it is preferably less dense and more viscous such that it can easily flow into the nooks and crevices which are regularly found there.

Finally, a third coupling region 36 includes optical coupling means. At the underside of a window transparent to optical beams, an index matching fluid can be applied. Index matching fluid is used in some optical systems to promote transmission of optical beams from one medium to another. In the present case, the skin presents problems for incident optical beams and its surface tends to cause undesirable scatter. This can be mitigated by introduction of a transparent fluid which has a similar index of refraction as flesh/water and the window described above.

As presented, these three coupling means, that is optical coupling means, acoustic coupling means, and spatial coupling means all can be formed upon a simple disposable element for use in conjunction with a well prepared measurement head at the test site on a tissue surface.

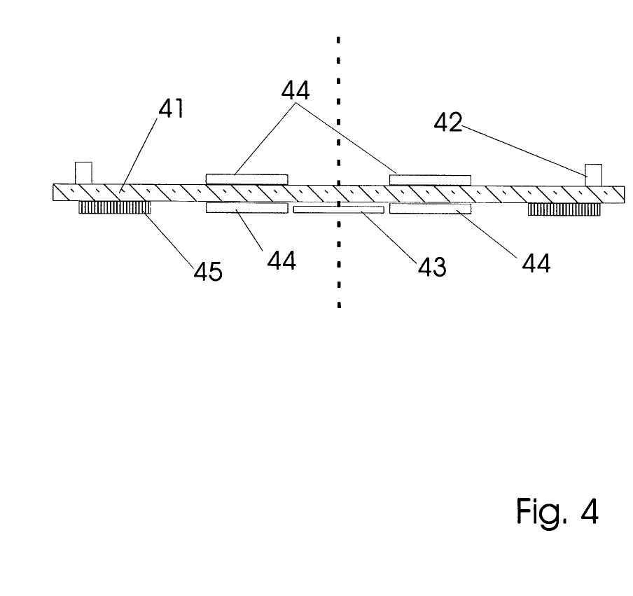

For clarity, the device is illustrated in drawing FIG. 4 in a cross sectional block diagram. While this diagram draws important relationships between major elements of the device, it is not intended as a mechanical or engineering drawing. The blocks are understood to represent what may actually be a detailed mechanical part.

FIG. 4 illustrates a preferred version of a disposable coupling disk of these inventions. In agreement with FIGS. 1-3, an inexpensive plastic disk 41 is a substrate upon which other coupling means are formed. Namely, an indexing and affixing means 42 is disposed at the disk periphery. The indexing means may be based upon mechanical interlock, adhesives, among others. Optical index matching fluid 43 is illustrated on the bottom side of the disk at the disk axis. The fluid may be used in conjunction with an optical window, anti-reflection coatings, lenses, et cetera; each of which may be formed directly into the plastic from which the disposable disk is made. In most of these inventions, optical sources preferably lie on a system axis and thus the optical coupling means is configured in accordance. Media to promote acoustic transmission 44, may be placed on either side of the disk at the locations indicated. One will fully appreciate that where a detector array occupies a specific shaped region, the media can be spread to cover corresponding regions on the surface of the coupling element. The drawing in cross section only shows the radial distribution but it is readily understood that a two dimensional shape which cannot be fully represented in the drawing may follow from the array design. Lastly, spatial coupling means 45 are also arranged at the disk periphery where they might provide a leverage advantage which advances their function of holding the disk to the tissue surface with security.

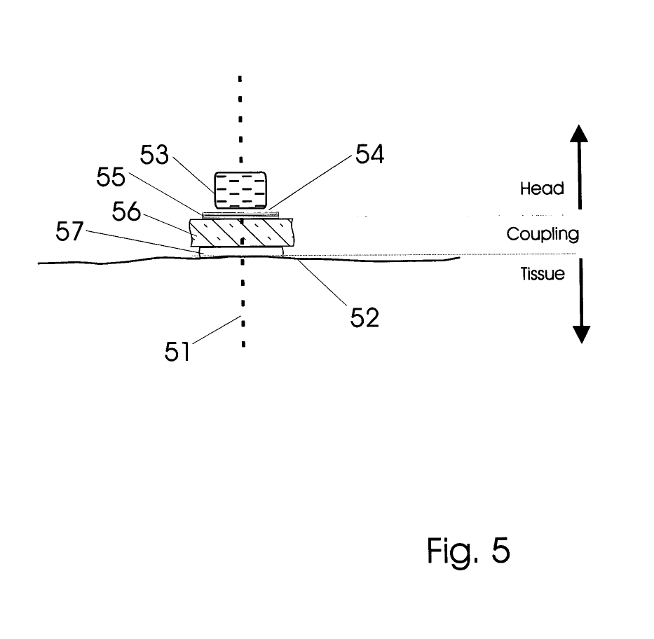

FIG. 5 is provided to show a detailed presentation of the optical coupling in one preferred version in isolation with respect to the other coupling means. Tissue 51 to be tested includes tissue surface 52 upon which lies a measurement head in conjunction with coupling means; in particular, optical coupling means. An optical source 53 which may include a laser or lasers is arranged above an air gap 54. Thin film anti-reflective coating 55 permits high transparency for light normally incident thereon. The light may propagate into the disposable element 56 and further into index matching fluid 57 which forms direct contact with the tissue. In versions where optical sources having special spectral characteristics, such as where Mid-IR wavelength light is used, the plastic disk may be replaced by a window which is transparent to longer wavelength infrared. A hole may be drilled into a disk on its axis and the hole is filled with a window blank for example. The window blank may be preformed with the thin film anti-reflection coating. This is a slight alternative to this otherwise preferred version and is to be considered included.

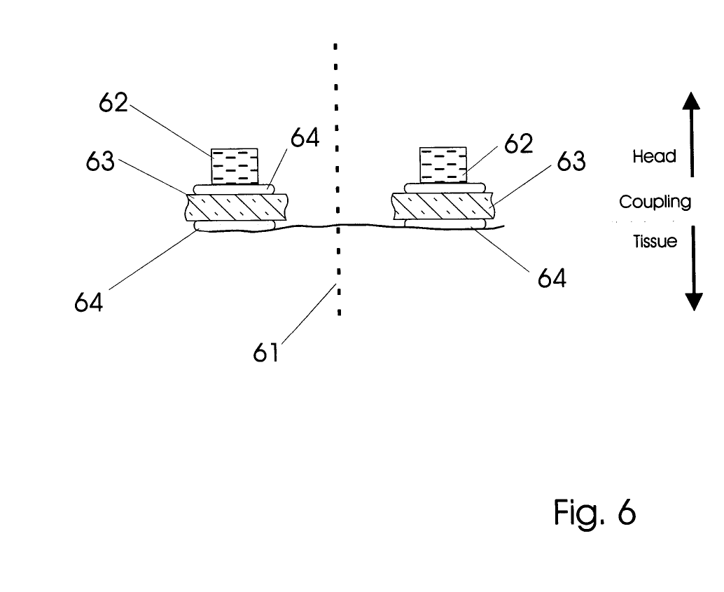

It is also instructive to view the acoustic coupling means in isolation. FIG. 6 presents an illustration of elements which form a preferred acoustic coupling system. Tissue test site 61 lies below tissue surface upon which sets an acoustic detection system comprising at least one microphone 62 and acoustic coupling means. Acoustic coupling means includes a disposable plastic disk 63 shown in cutaway portions only. Both atop and below the disk on its surface is acoustic transmission media which may preferably be a gel or dense liquid material. Acoustic waves in the tissue propagate towards the tissue surface where the energy is easily passed from the skin into the acoustic transmission gel which tends to fill the irregular forms of the skin surface. Acoustic energy further propagates through the disk and into the acoustic transmission media on the top of the disk. Finally, the acoustic energy is passed into the microphone elements where it is converted into an electronic signal which may be processed.

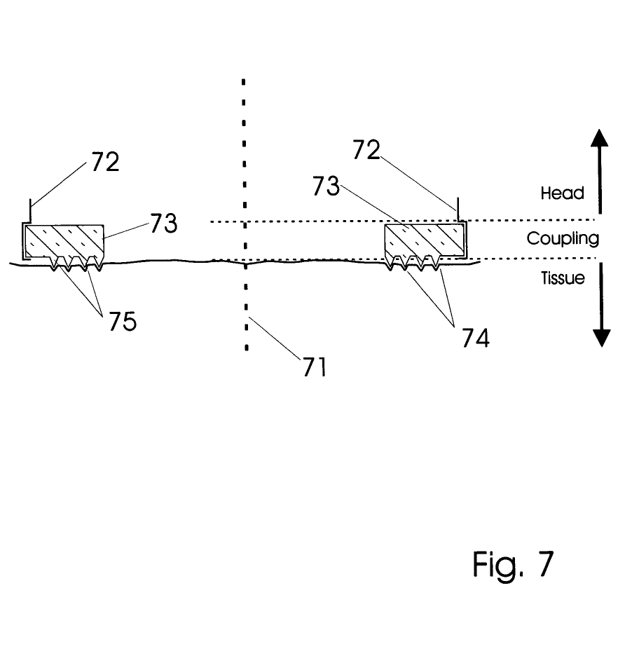

FIG. 7 shows a detailed presentation of mechanical coupling in one preferred version. Tissue site 71 is addressed by a measurement head which includes spatial coupling means. Spatial coupling means of preferred versions include a mechanical interlock. Measurement heads include at their periphery a mechanical interlock 72. These may be embodied in many forms such as matching threaded elements, pressure ‘snap-in’ systems, et cetera. In the illustrated system, a disposable coupling disk 73 is a formed of a size which precisely fits within interlock 72. With pressure, the disk may be removed from and replaced into its receiving cavity formed by the interlocking element which may be formed integrally with the measurement head. Spatial coupling means further includes systems for holding the device steady to the tissue at a plurality of points 74 on the skin surface. Micro mechanical ‘teeth’ 75 forming in the plastic disk tend to bite into the skin surface and provide a non-slip hold thereon. In this way, a measurement head in cooperation with coupling means is held to a particular position on the tissue which defines the test site.

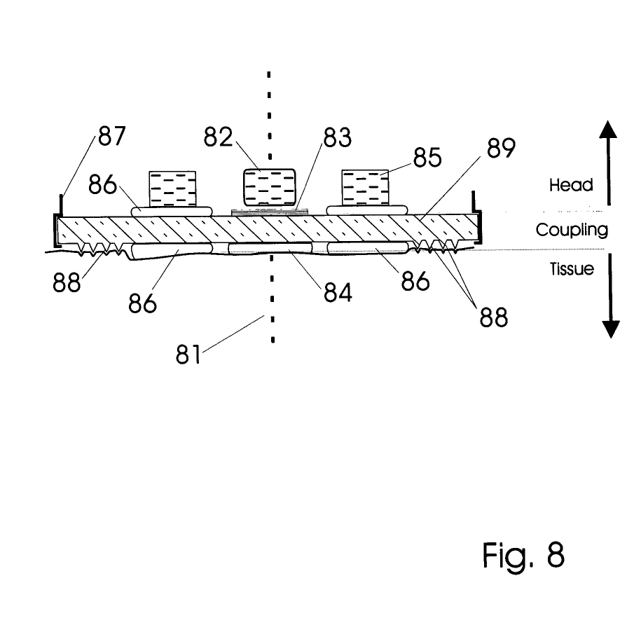

In review, one might consider these coupling systems altogether provided in a single system. FIG. 8 illustrates a disposable coupling element which is comprised of optical coupling means, acoustic coupling means, and mechanical coupling means. More precisely, tissue site 81 lies beneath optical source 82, thin film anti-reflecting coating 83, optical index matching fluid 84, microphones 85, acoustic transmission media 86, mechanical interlock 87, gripping teeth 88. These are built together upon a simple plastic disposable disk substrate 89 which supports it all as a system.

While the presented version and its minor variations were described in detail, it is useful to consider further variation. The nature of these inventions permits great variety of arrangement while accomplishing identical tasks and function. Thus, it is important to present a spectrum of these to more fully illustrate the true breadth of the inventions.

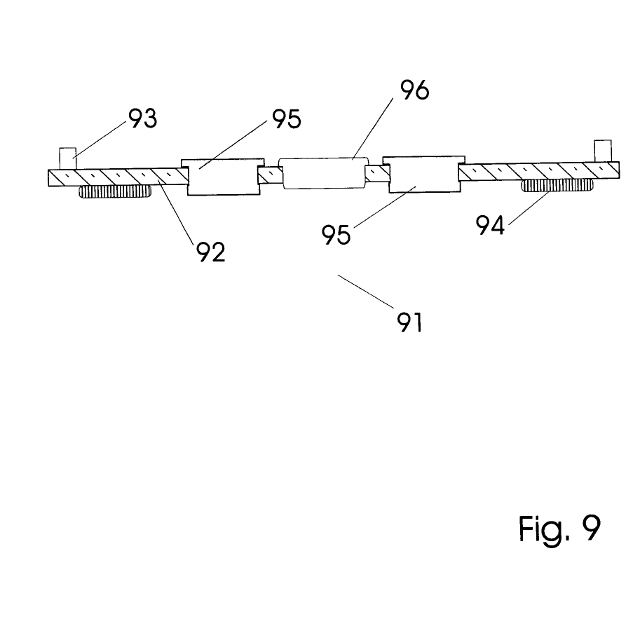

FIG. 9 illustrates a very important deviation. Some abbreviated versions include those where the disk is prepared with ‘cutouts’ appropriately spaced about the disk. These ‘cutouts’ are holes which extend through the disk and into which complementary shaped elements may be inserted. In one version, tissue site 91 is engaged by disposable coupling element having disk 92 including indexing and affixing means 93 and gripping system 94. A specially shaped element of high density material serves as an acoustic conduit 95. For example, hard rubber may be precisely shaped to fill the cutout in the disk and provide a direct path from the tissue to the microphones of a detector array. Similarly, a special window 96 having preferred optical transmission properties may be pushed into a carefully prepared receiving hole in the disk. In this way, a special version of a disposable coupling system is formed. Both the acoustic conduit member and the optical window may be enhanced with liquid and gels to improve coupling to the tissue surface. Further, the optical window might include at its top surface a relief pattern in a prescribed form to effect lensing action there. A Fresnel type lens can be formed in an etching process to promote focusing of light beams which pass the surface.

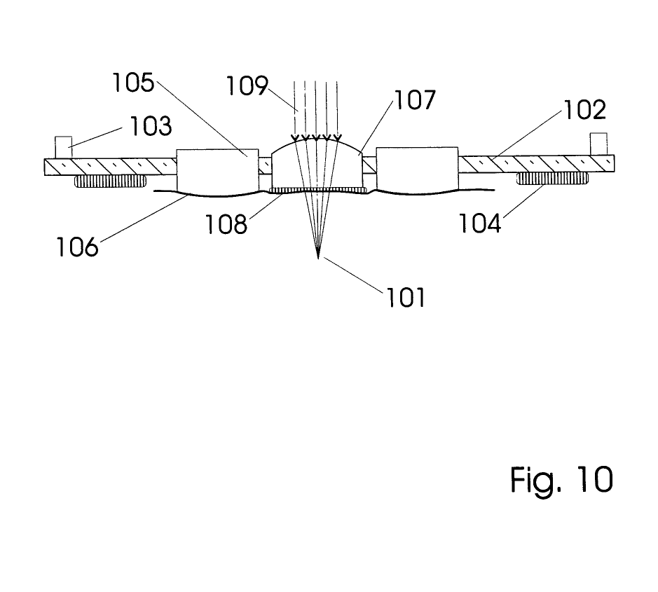

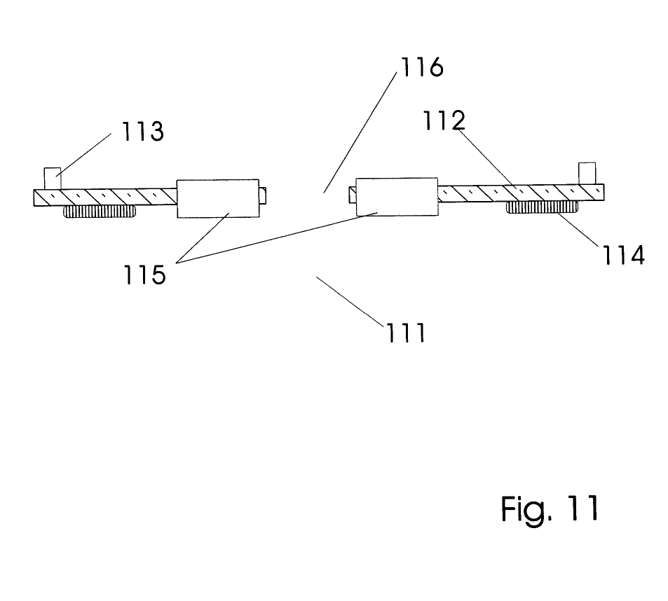

FIG. 10 illustrates a special version where an optical lens forms an immersion lens relationship with the tissue test site 101. Disposable disk 102 includes index and affixing means 103, and grippers 104. Special acoustic conduits 105 are provided with a curved contact surface 106 which operate to engage skin surface and pass acoustic energy without need for a gel interface material. This is partly due to the curved surface which tends to displace the skin and tissue into a cooperative shape. In addition, refraction at the surface acts as a lens to gather and concentrate more acoustic energy. Further, lens 107 is specially arranged to form an immersion lens with the tissue as part of the lens. That is, coupling fluid 108 makes the junction seamless to a beam entering the tissue and rays 109 come to a focus in accordance with principles of common immersion lenses; however in this case tissue becomes part of the lens.

In yet another version, an optical port is formed in the center of a disposable coupling element which additionally carries acoustic and spatial coupling means. Test site 111 lies beneath coupling disk 112 having spatial coupling means 113 and 114 and acoustic coupling means 115. Optical port/aperture 116 is merely a hole cut into the disk which allows optical beams to readily pass therethrough. In this case, ‘optical coupling means’ is simply a clear path to the tissue through the coupling disk.

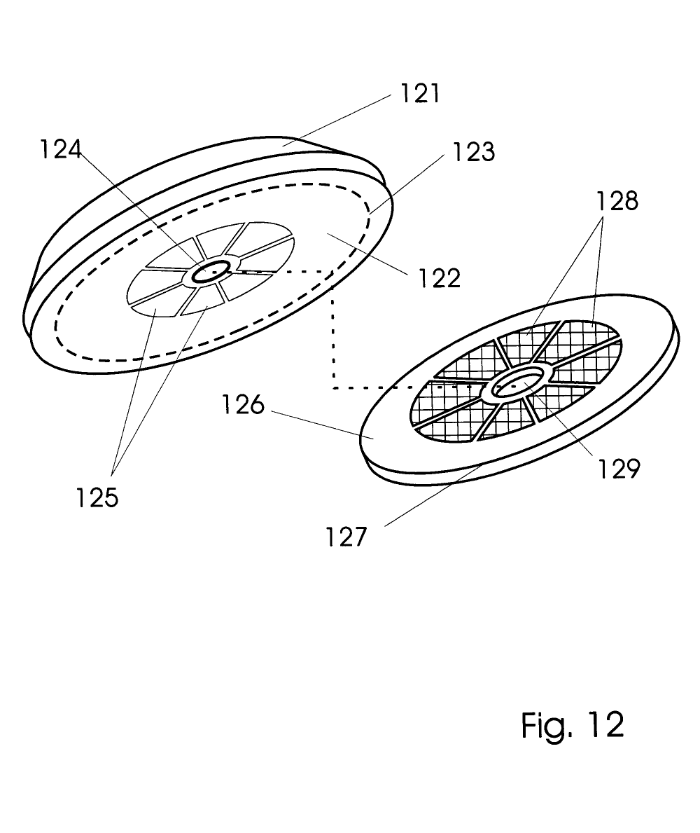

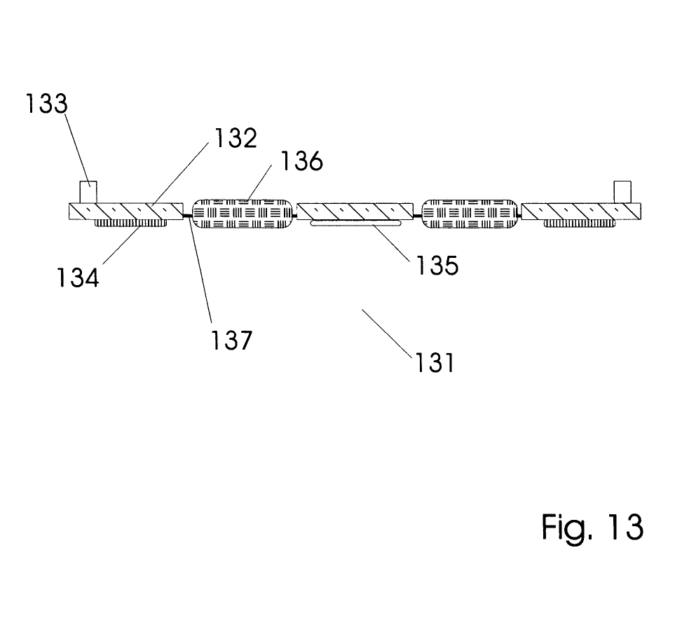

FIG. 12 illustrates another important variation with respect to acoustic coupling. A measurement head 121 has an underside surface 122 having therein a surface relief ridge 123, and an optical source 124 and microphone array 125. Coupling element 126 is disk shaped and fits precisely at its periphery 127 within a partial cavity formed by the ridge 123 and the undersurface 122. The coupling disk can be formed with a cutout regions which correspond to the shapes of individual detector elements, microphones, in the detector array. Into those cutouts, a very thin and lightweight mesh 128 can be inserted. The mesh is useful to support media such as gel which provides good acoustic transmission there through. The gel material makes good acoustic contact with surfaces like skin and promotes transfer of energy from the tissue to a detector. The mesh tends to hold the gel and prevent it from flowing away from the region of the cutout. This concept is more fully illustrated in consideration of the cross section view of FIG. 13. Test site 131 lies beneath disposable coupling element made of a plastic disk 132, spatial coupling means 133 and 134, optical coupling 135. Gel 136 is held by mesh 137 in cutout regions distributed appropriately in accordance with a prescribed pattern. When the coupling element is set to the skin surface, the gel comes into intimate contact with the skin and further to individual detectors. Acoustic energy from the tissue is isolated in the gel of one particular region and passed to the appropriate detector. Acoustic energy is not permitted to pass from a first cutout to another in any appreciable amount due to the natural acoustic path provided by the gel directly to the acoustic transducer/microphone.

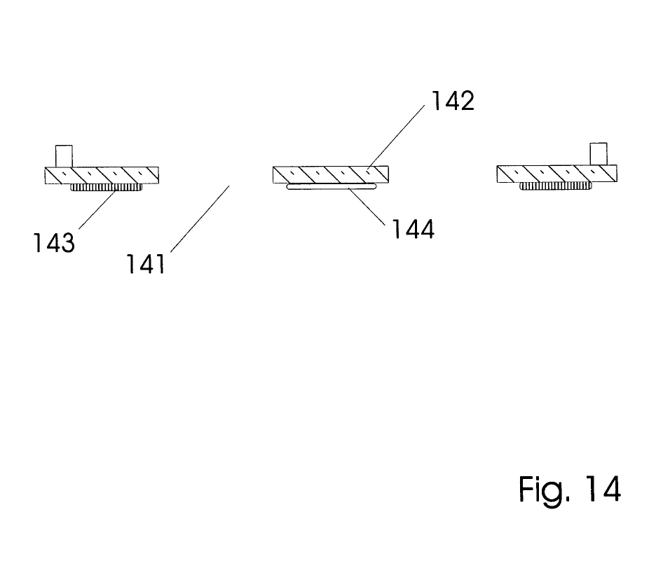

FIG. 14 illustrates another version where cutouts are provided without a mesh therein. Rather, the cutouts 141 accommodate acoustic detectors which protrude down from a measurement head and extend past the disk 142 bottom surface to come directly into contact with the skin surface. Spatial coupling 143 and optical coupling 144 might be effected in the normal fashion without interference from such arrangement.

The examples above are directed to specific embodiments which illustrate preferred versions of devices of these inventions. In the interests of completeness, a more general description of devices and the elements of which they are comprised as well as methods and the steps of which they are comprised is presented herefollowing.

In restatement these inventions include photoacoustic biometric measurement systems having disposable couplings. The couplings include a thin substrate element having thereon an optical coupling means and an acoustic coupling means. The coupling systems are arranged to pass optical and acoustic energy from corresponding transducers to/from human flesh. In most preferred versions said substrate is a disk shaped element which fits or is affixed onto a measurement head. The coupling is arranged to reduce movement between said coupling and a tissue test site.

Optical couplings are preferably arranged at the center of the disk and sometimes includes an index matching fluid or optically compatible gel in conjunction with other optical elements, a thin film anti-reflection coating, for example. These couplings may also include a lens operable for focusing and concentrating light into an abbreviated space. An optical coupling may merely be an aperture cut into said substrate.

Acoustic coupling sometimes includes an acoustic conduit. Some versions include an acoustic conduit in contact with tissue via an acoustic coupling gel. An acoustic conduit may be a dense material such as hard rubber pressed fit into receiving holes in a substrate. These may include curved contact surfaces suitable for being in direct contact with the skin surface. Acoustic couplings might include mesh elements within apertures cut into substrates. The mesh may further include acoustic coupling gel thereon.

One will now fully appreciate how disposable couplings for in vivo photoacoustic measurements may be arranged to effect highly efficient systems. Although the present invention has been described in considerable detail with clear and concise language and with reference to certain preferred versions thereof including the best mode anticipated by the inventor, other versions are possible. Therefore, the spirit and scope of the invention should not be limited by the description of the preferred versions contained therein, but rather by the claims appended hereto

1) In photoacoustic biometric measurement systems, disposable couplings comprising: a substrate element having thereon an optical coupling means arranged to efficiently pass optical energy from at least one optical source into human flesh; and an acoustic coupling means arranged to efficiently pass acoustic energy to and from at least one acoustic transducer into human flesh.

2) Disposable couplings of claim 1, said substrate is a disk shaped element having axial symmetry and is arranged to cooperate with a measurement head having optical and acoustic transducers.

3) Disposable couplings of claim 1, further comprising spatial coupling means arranged on either surface of said substrate to promote spatial stability and reduce movement between said coupling and a tissue test site.

4) Disposable couplings of claim 1, further comprising spatial coupling means arranged on either surface of said substrate to temporarily affix said substrate to said measurement head.

5) Disposable couplings of claim 3, said coupling being disposed at or near the periphery of said substrate.

6) Disposable couplings of claim 2, optical coupling means is arranged on a symmetry axis of said disk.

7) Disposable couplings of claim 6, said optical coupling includes an index matching fluid or optically compatible gel.

8) Disposable couplings of claim 7, said optical coupling further includes a thin film anti-reflection coating between an optical source and tissue at a test site.

9) Disposable couplings of claim 7, said optical coupling further includes a lens operable for focusing and concentrating light into abbreviated tissue space.

10) Disposable couplings of claim 9, said lens is immersion type lens with live tissue as part of lensing medium.

11) Disposable couplings of claim 6, said optical coupling is an aperture cut into said substrate.

12) Disposable couplings of claim 2, said acoustic coupling means is further comprised of at least one acoustic conduit, said acoustic conduit operable for providing communication between tissue and a microphone.

13) Disposable couplings of claim 12, said acoustic conduit includes holes or vias through said substrate.

14) Disposable couplings of claim 12, said acoustic conduit is in contact with tissue via an acoustic coupling gel.

15) Disposable couplings of claim 13, said acoustic conduit is a dense material press fit into receiving holes in the substrate.

16) Disposable couplings of claim 12, said dense material includes a curved contact surface suitable for being directly in contact with skin surface.

17) Disposable couplings of claim 12, said acoustic coupling means includes a mesh element within aperture cut into said substrate.

18) Disposable couplings of claim 17, said mesh element includes acoustic coupling gel thereon.

19) Disposable couplings of 17, said substrate includes multiple regions distributed about an annulus.

20) Disposable couplings of claim 2, said acoustic coupling means includes a plurality of apertures in the substrate which permit direct contact between at least one microphone and tissue being tested via said holes in substrate.

21) A photoacoustic biometric measurement system comprising:
a durable wearable portion and a disposable, lifetime sensitive, coupling element of claim 1.

22) A photoacoustic biometric measurement systems of claim 21, said durable wearable portion comprising: an optical source and an acoustic transducer.

23) A photoacoustic biometric measurement systems of claim 21, said disposable portion comprising: a thin substrate element; an optic coupling means arranged to efficiently pass optical energy from at least one optical source into human flesh; and an acoustic coupling means arranged to efficiently pass acoustic energy to and from at least one acoustic transducer into human flesh, said thin substrate being arranged to have thereon said acoustic coupling means and said optic coupling means.
